(19) United States
(12) Patent Application Publication
(10) Pub. No.: US 2010/0305443 A1
(43) Pub. Date: Dec. 2, 2010

(54) APPARATUS AND METHOD FOR MEDICAL SCANNING

(76) Inventors: Stewart Gavin Bartlett, Southern Australia (AU); Paul James Hirschausen, Southern Australia (AU)

Correspondence Address: Intellectual Property Dept.;Dewitt Ross & Stevens SC, 2 East Mifflin Street, Suite 600, Madison, WI, 53703-2865, US

(21) Appl. No.: 12/675,473

(22) PCT Filed: Aug. 29, 2008
(86) PCT No.: PCT/AU2008/001278

§ 371 Date: Feb. 26, 2010

(30) Foreign Application Priority Data
Aug. 31, 2007 (AU) 2007904741

(51) Int. Cl. A61B 8/14

(52) U.S. Cl. 600/443; 600/459

(57) ABSTRACT

A hand held ultrasound imaging system with a probe unit having a transducer being adapted to transmit and receive ultrasonic signals and an orientation sensor adapted to sense the rotation of the probe unit, the output of the transducer and of the sensor being combined to produce a set of scanlines having a series of intensity values and a rotation value, the scanlines then being processed to produce a raster image for display on a display unit.

### TECHNICAL FIELD

The present invention relates to an improved method and apparatus for ultrasound scanning of a subject, providing cost and range of application advantages. The method has particular application to the field of hand-held ultrasound equipment.

### BACKGROUND ART

Ultrasound was first investigated as a medical diagnostic imaging tool in the 1940's. This was based on the use of A-mode (amplitude mode) ultrasound, which is a form of echo ranging. This simply gives a plot of returned echo intensity against time, which, by knowing the speed of sound in the target media, gives the distance of the features returning the echo from the transducer. In order to obtain valid information from such a scanline it is necessary that the direction of the transmitted ultrasound beam be constant and known.

In order to provide an imaging system, it is necessary to insonify a larger area, at least a two dimensional slice of the target. It is also necessary to receive returned echoes from this area and to display this information in correct spatial relationship.

Since the only information received by an ultrasound transducer is echo intensity over time, spatial information can most easily be added by knowing the direction from which the echo was received. This means knowing the position and orientation of the transducer at all times and this was most easily achieved by controlling the movement of the transducer.

This led to B-mode (brightness mode) scanning, where the ultrasound output is pulsed and the transducer is mechanically scanned over the target. The transducer detects the echo from each pulse as intensity versus time, called a scanline. The scanlines are displayed with brightness being proportional to echo intensity, thus forming an image.

In the early 1950's Wild and Reld constructed a B-mode scanning system using a mechanically mounted rotating transducer.

Ultrasound technology developed significantly in the 1960's with the development of articulated arm B-mode scanners by Wright and Meyerdirk. Articulated arm scanners, also known as static mode scanners, connect the ultrasonic transducer to a moveable arm, with movement of the arm mechanically measured using potentiometers. The articulated arm also ensures that the degree of freedom of movement of the transducer is limited to a defined plane. This allowed the position of the transducer to be known with considerable accuracy, thus allowing the scanlines recorded by the transducer to be accurately located in space relative to each other for display.

Static mode ultrasound scanners were in wide use until the early 1980s. The static mode scanners were large cumbersome devices, and the techniques used are not readily suited to a handheld ultrasound system.

In the mid 1970's real-time scanners were developed where an ultrasonic transducer was rotated using a motor. Krause (U.S. Pat. No. 3,470,868—Ultrasound diagnostic apparatus) describes an invention where a motor rotates an ultrasonic transducer in order to produce images in real-time.

Motor driven transducers removed the need for precise knowledge of the position of the transducer housing, since the operator needed only to hold the transducer housing still and the motor would sweep the transducer rapidly to produce a scan arc. This resulted in an evenly distributed set of scanlines, in a single plane, whose spatial relationship was known because the sweep characteristics were known.

These devices brought their own problems. The motor driving circuitry added size, power consumption, complexity and cost to the device. Additionally, the motor itself and associated moving parts reduced the reliability of the device.

A solution to these problems has been sought in electronic beam steering transducers. Wilcox (U.S. Pat. No. 3,881,466) describes an invention consisting of a number of electronic crystals where the transmitting pulse can be delayed in sequence to each crystal and thus effect an electronic means to steer the ultrasound beam. The basic technique is still in wide use today, with nearly all modern medical ultrasound equipment using an array of ultrasonic crystals in the transducer. The early designs used at least 64 crystals, with modern designs sometimes using up to a thousand crystals or more.

Electronic beam steering removes the need for a motor to produce real time images. The scanlines resulting from the use of an array transducer are contained within a defined plane, or in the case of 2-D arrays within a defined series of planes. The scanlines may therefore be readily mapped onto a flat screen for display.

However, the cost of producing transducers with arrays of crystals is high. There is also a high cost in providing the control and processing circuitry, with a separate channel being required for each crystal. The transducers are usually manually manufactured, with the channels requiring excellent channel to channel matching and low cross-talk. The power consumption for electronic systems is also high, and is generally proportional to the number of channels being simultaneously operational.

In parallel, solutions to the problem of tracking a transducer without using articulated arms were pursued. These involved tracking the transducer, or a component with a fixed relationship to the transducer, in relation to an external reference frame. These generally involved electromagnetic tracking using one or more fixed transmitters separate from the transducer unit, and a receiver on the transducer unit. Visual tracking using cameras was also employed.

These all suffered from the need to establish the frame of reference, in some cases only being of use in specifically equipped rooms. They also suffered from the problem of interference with the tracking signals by people and equipment moving in the field of reference. These problems, in particular made these systems unsuitable for hand-held use.

Much of the prior art in ultrasound technology is directed to improving the performance of ultrasound systems enabling them to be used for an ever increasing range of diagnostic applications. The result has seen significant advances in ultrasound systems with transducers using ever increasing numbers of crystals, and host systems with ever increasing processing power. The result has seen systems with 3D and real-time 3D (or 4D) capability.

These high cost, high power consumption devices are unsuitable for broad point-of-care application outside of specialist sonography facilities. In particular, these systems are unsuitable for application to hand-held devices.

### DISCLOSURE OF THE INVENTION

In order to put ultrasound capability into the hands of point of care personnel, factors of cost, size, form factor and usability need to be considered. Power usage is also important, since a hand held device is most conveniently battery powered. A simple, single beam transducer, manually swept over a region of interest provides advantages.

Therefore, in one form of this invention although this may not necessarily be the only or indeed the broadest form of this there is proposed an ultrasound imaging system adapted for hand held use including

a probe unit having a transducer in a fixed spatial relationship with the probe unit, said transducer being adapted to transmit and receive ultrasonic signals,
an orientation sensor adapted to sense the rotation of the probe unit about at least one axis,
electronics adapted to apply a pulsed voltage to the transducer and to process the electrical output signal of the transducer and of the sensor to produce a plurality of scanlines each having a series of intensity values and a rotation value,
a processor adapted to process the scanlines to produce a raster image,
a display adapted to display the resultant raster image.

In preference the sensor is an inertial sensor.

An advantage of using an inertial sensor is that it is self contained. The sensor can be fully contained in the probe unit, without the need for an external reference.

In a further form the invention may be said to lie in a method of ultrasound imaging including the steps of

applying a probe unit including an ultrasound transducer adapted to transmit and receive ultrasonic signals into and from a target body,
transmitting ultrasonic pulses into said target body and receiving return signals
rotating said probe unit substantially in a single plane such that a two dimensional section of the target body is scanned
using a sensor to provide rotation information about the rotation of the probe unit about at lest one axis,
combining the return signals with the rotation information to produce scanlines,
processing the scanlines to produce a raster image,
displaying the raster image on a display.

It has always been considered in the prior art that inertial sensors suffer from calibration problems which render them unsuitable for this use. However, the apparatus and method of the invention allow medically useful data to be extracted without calibration being a significant issue.

In preference, the sensor includes a gyroscope.

In preference, the sensor includes two or more orthogonally mounted gyroscopes.

In preference, the sensor includes an accelerometer.

In preference, the sensor includes two or more orthogonally mounted accelerometers.

In preference, the rotation is relative to a selected scanline.

The method and apparatus of the invention allow very useful information can be obtained by sensing only orientation and/or changes in orientation of the probe unit, without sensing linear displacement.

An advantage of the invention is that a low cost transducer adapted to scan only in a single direction at any instant and low cost orientation detection devices can be used to produce diagnostically vary useful 2D tomographic images of a body to be scanned.

### BRIEF DESCRIPTION OF THE DRAWINGS

FIG. 1 illustrates an ultrasonic scan system including an embodiment of the invention;

FIG. 2 illustrates a probe unit showing the relationship to the orientation sensor;

FIG. 3 illustrates a block diagram of a hand held ultrasound system of the invention;

FIG. 4 illustrates a time gain compensation diagram;

FIG. 5 illustrates a scan data set;

FIG. 6 illustrates a partial block diagram of the functional blocks of a probe unit controller;

FIG. 7 illustrates an ultrasound scan space, with the pixel grid of a display overlaid upon it.

FIG. 8 illustrates a partial ultrasound scan space, with the pixel grid of a display overlaid upon it, illustrating scanline/rowline intersection;

FIG. 9 illustrates an ultrasound pulse and an exemplary echo return;

FIG. 10 illustrates the selection of a scan data point as a pixel value.

FIG. 11 illustrates an example of an idealised scan and its practical realisation in a system of the invention.

FIG. 12 illustrates an enveloping function applied to a return signal.

### BEST MODE FOR CARRYING OUT THE INVENTION

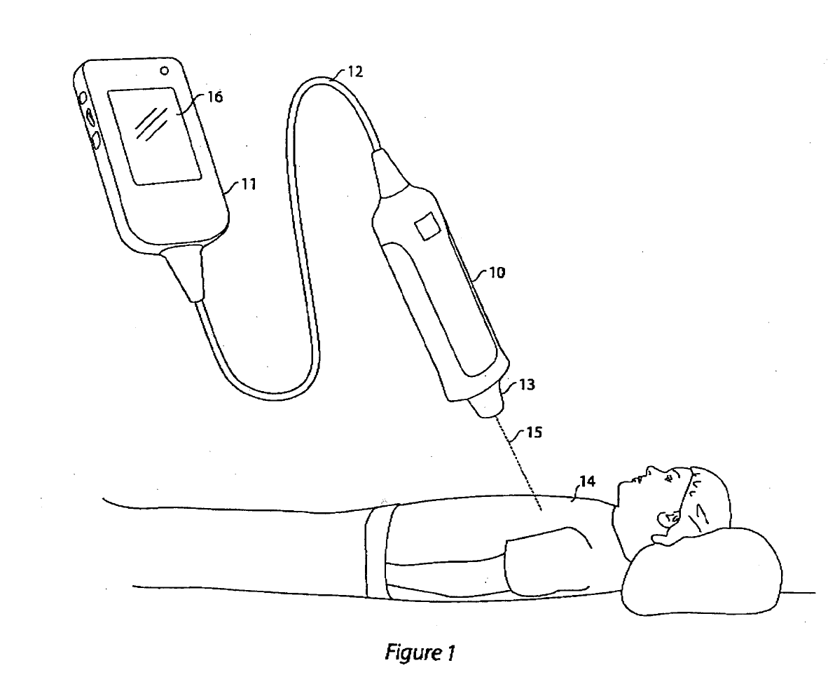

Referring now to FIG. 1, there is illustrated an ultrasonic scan system according to an embodiment of the invention. There is a hand held ultrasonic probe unit 10, a display and processing unit (DPU) 11 with a display screen 16 and a cable 12 connecting the probe unit to the DPU 11.

The probe unit 10 includes an ultrasonic transducer 13 adapted to transmit pulsed ultrasonic signals into a target body 14 and to receive returned echoes from the target body 14.

In this embodiment, the transducer is adapted to transmit and receive in only a single direction at a fixed orientation to the probe unit, producing data for a single scanline 15.

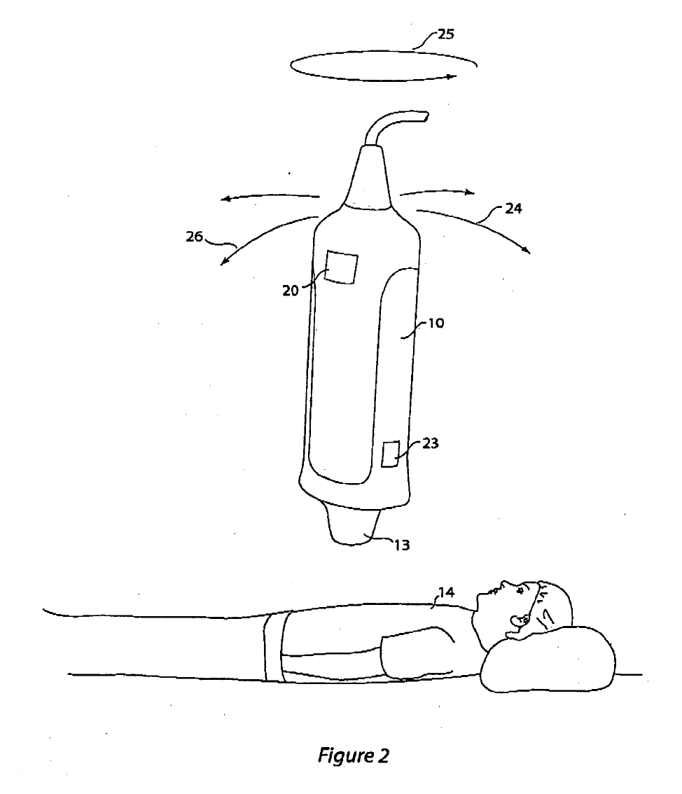

As shown in FIG. 2, the probe unit further includes an orientation sensor 20 capable of sensing orientation or relative orientation about one or more axes of the probe unit. Thus, in general, the sensor is able to sense rotation about any or all of the axes of the probe unit, as indicated by rotation arrows 24, 25, 26.

The sensor may be implemented in any convenient form. In an embodiment the sensor consists of three orthogonally mounted gyroscopes. In further embodiments the sensor may consist of two gyroscopes, which would provide information about rotation about only two axes, or a single gyroscope providing information about rotation about only a single axis.

Since the distance between the mounting point of the sensor 20 and the tip of the transducer 13 is known, it would also be possible to implement the sensor with one, two or three accelerometers.

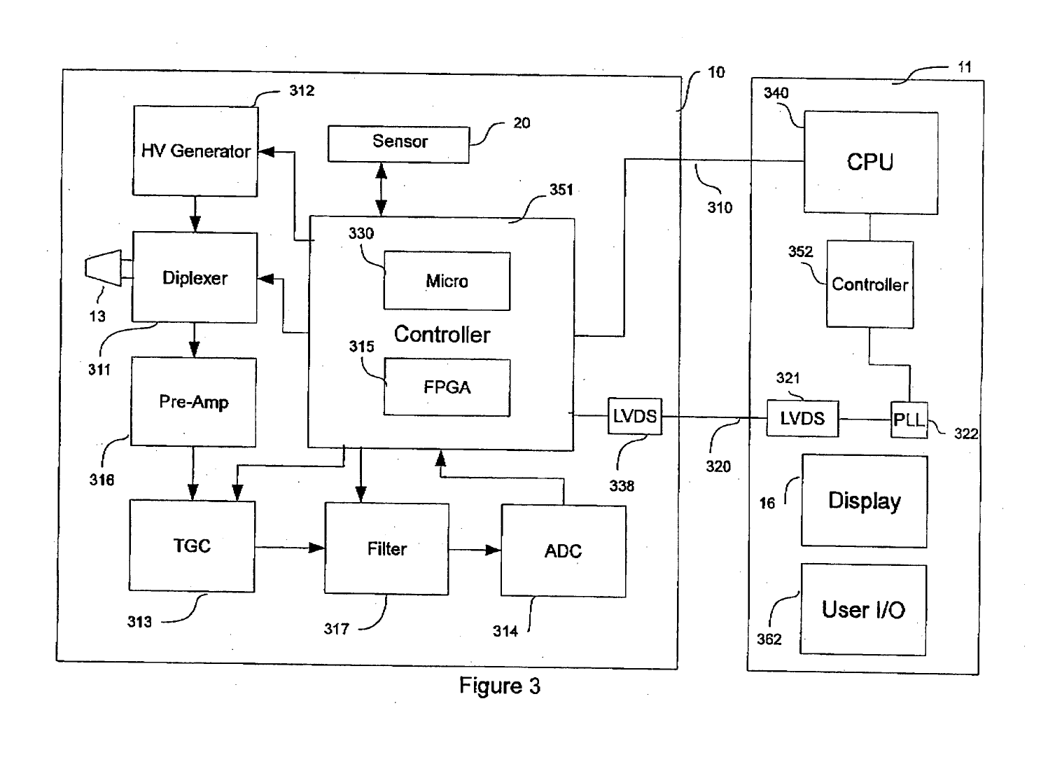

A block diagram of the ultrasonic scan system is shown in FIG. 3. There is a probe unit 10 and a DPU 11. The probe unit includes a controller 351 which controls all of the functions of the probe. In this embodiment, the controller is implemented as a combination of a field programmable gate array (FPGA) 315 and a microcontroller 330.

The DPU includes a main CPU 340 and a communications controller 352.

The probe unit 10 communicates with the DPU 11 via a low speed message channel 310 and a high speed data channel 320. The message channel is a low power, always on connection. In an embodiment, it is implemented as a direct connection between the microcontroller 330 on the probe unit and the main CPU 340 of the DPU. In this embodiment, it is implemented using I²C bus technology.

The data channel is a higher speed and hence higher power consumption bus which is on only when required to transmit data from the probe unit to the DPU. In this embodiment, it is implemented as a low voltage, differential signal (LVDS) bus. In this embodiment, it is a single channel. Multiple channels may be used in other embodiments, to carry higher data rates or separate sensor channels.

The probe unit includes a transducer 13 which acts to transmit and receive ultrasonic signals. A diplexer 311 is used to switch the transducer between transmit and receive circuitry.

On the transmit side the diplexer is connected to high voltage generator 312, which is controlled by controller 351 to provide a pulsed voltage to the transducer 13. The transducer produces an interrogatory ultrasonic pulse in response to each electrical pulse.

This interrogatory pulse travels into the body and is reflected from the features of the body to be imaged 14 as an ultrasonic response signal. This response signal is received by the transducer and converted into an electrical received signal.

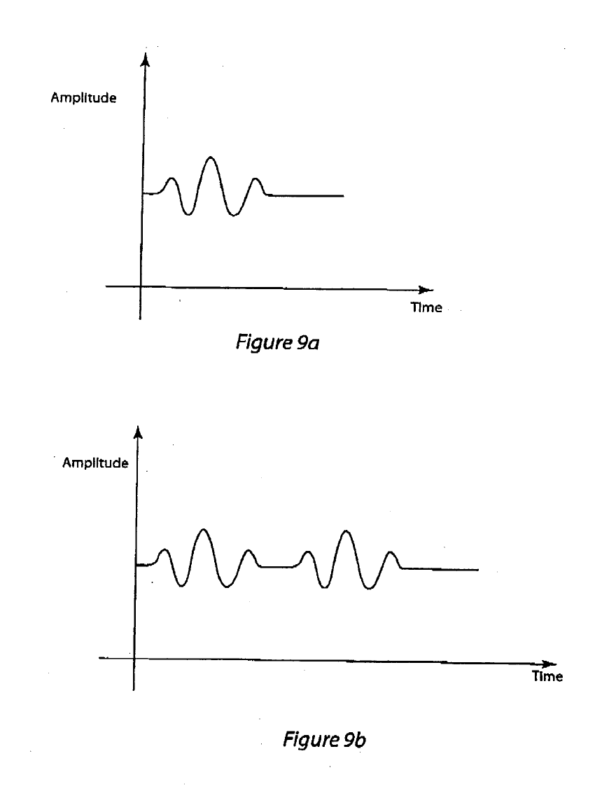

A plot of the transducer pulse in the time domain is shown in FIG. 9a. An exemplary response signal is shown in FIG. 9b. This response signal is the intensity value of the returned echo.

The depth from which the echo is received can be determined by the time delay between transmission and reception, with echoes from deeper features being received after a longer delay. Since the ultrasound signal attenuates in tissue, the signal from deeper features will be relatively weaker than that from shallower features.

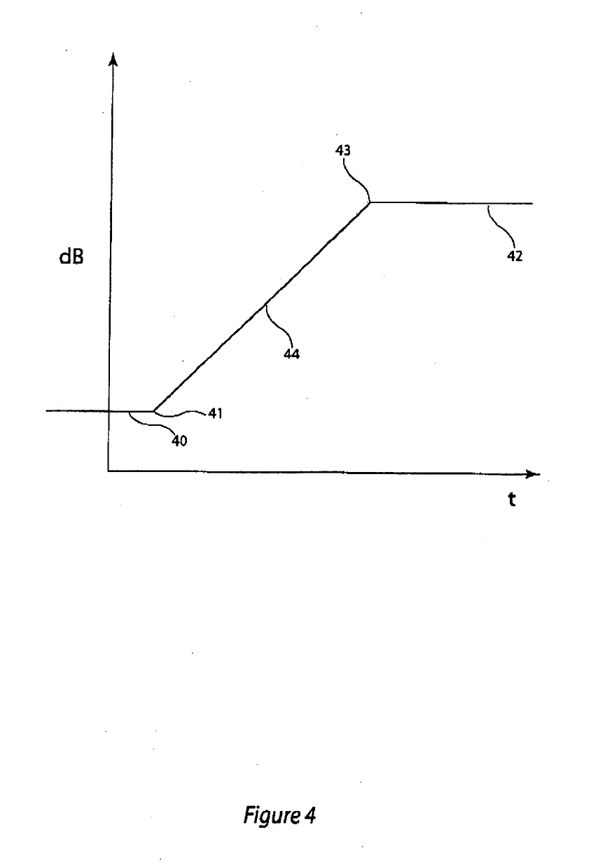

The diplexer 311 connects the electrical receive signal to time gain compensation circuit (TGC) 313 via a pre-amp 316. The TGC applies amplification as shown in FIG. 4, to the received signal. This shows a plot of amplification against time to be applied to the returned echo for each pulse. The characteristics of the amplification are selected to compensate for the depth attenuation, giving a compensated receive signal where the intensity is proportional to the reflectiveness of the feature which caused the echo. In general, the amplification characteristics may take any shape.

This compensated signal is passed to an analogue to digital converter (ADC) 314, via an anti-aliasing filter 317. The output of the ADC is a digital data stream representing the intensity of the received echoes over time for a single ultrasonic pulse.

There is an orientation sensor 20 which is adapted to provide information about angular rotation of the probe unit.

The DPU includes a touchscreen user interface device 16. This gives the user control of a user interface which allows parameters for an ultrasound scan to be set. Further user input devices 362 may be provided. These include but are not limited to, a scroll wheel, numeric or alpha numeric keypad and voice recognition means.

The parameters which may be set may be any variable affecting the ultrasound. They include the sample rate for the ADC, the number of values to be taken, the length of a scan in time or in angle traveled by the probe unit.

The set up parameters for the TGC as shown in FIG. 4 may also be set. These include the initial amplification 40, and the time 41 to which this should be applied, and the final amplification 42 and the time 43 at which this should be reached. This defines the slope of the TGC ramp 44. This control allows the TGC to be set appropriately for the attenuation profile of the material being imaged.

Returning to FIG. 2, in use, a user applies the probe unit 10 to a body to be imaged 14. The communication button 23 is pressed to initiate a scan. The button press is detected by the microcontroller and communicated to the DPU via the message channel 310.

The DPU responds with a message which includes the parameters which have been selected for the scan. The controller 351 controls the high voltage driver to produce the required pulse sequence to be applied via the diplexer to the transducer in order to perform a scan according to the parameters set by the user, or set as defaults in the DPU.

The user rotates the probe as required to sweep the ultrasound beam over the desired area, keeping linear displacement to a minimum.

In embodiments where rotation about all axes is not sensed, the user will also keep rotation about unsensed axes, that is axes about which rotation is not detected by the sensor of the embodiment, to a minimum.

At the same time, data is received from the orientation sensor 20. This is the rotation about the sensed axes of the probe unit. It may be the angular change in the position of the probe unit since the immediately previous transducer pulse, or the orientation of the probe unit in some defined frame of reference. One such frame of reference may be defined by nominating one transducer pulse, normally the first of a scan sequence, as the zero of orientation.

The sensor data and the response signal are passed to the controller 351 and in particular the field programmable gate array (FPGA) 315 where they are combined to give a scanline. A scanline is a dataset which comprises a sequential series of intensity values of the response signal combined with orientation information. A scan dataset is a plurality of sequentially received scanlines.

A scan data set is built up by a user rotating the probe unit about at least one sensed axis while keeping the positional displacement to a minimum. The high voltage generator 312 continues to provide the pulsed voltage to the transducer under control of the microcontroller and each pulse results in a scanline.

More than one transducer may be used, such that more than one scanline is produced at a time. In an alternative embodiment, three transducers are mounted at a fixed angle of fifteen degrees to each other. Other numbers or transducers and angles of separation are possible. All three transducers are driven together. The angle of orientation received from the orientation sensor is adjusted by the amount of the angular offset of the transducers from each other in order to produce scanlines with consistent angular data. This results in a denser coverage of the area of interest, or allows for a slower pulse rate of the transducer, or a faster movement of the probe for the same density of coverage.

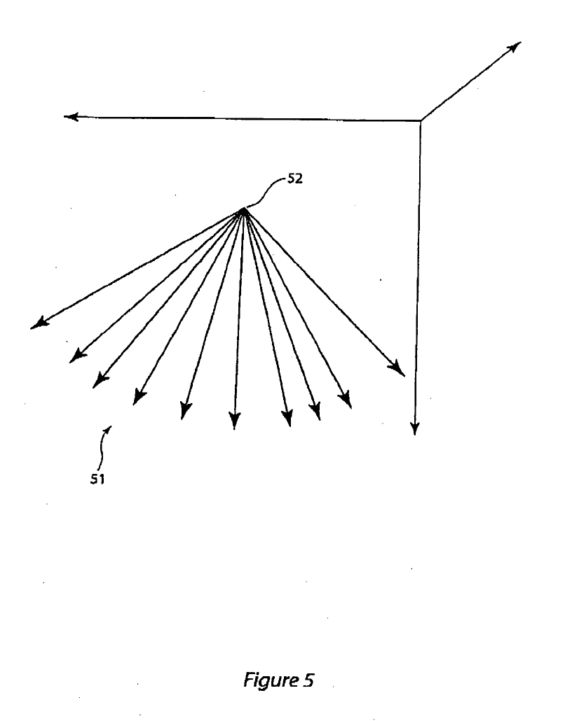

The result is a scan data set, as illustrated in FIG. 5. The scan data set may be seen to consist of a series of scanlines 51, each of which has an origin 52, a direction, and a depth. Taken together, these constitute the echo data for some geometric region in the target body. Since only orientation data is collected, the origins of all of the scanlines are co-incident, since no information about any linear displacement which may have occurred is available. They are not, in general, co-planar.

In embodiments where rotation about only a single axis is sensed by the sensor, the scanlines will be co-planar, since no information about rotation out of the plane orthogonal to the sensed axis will be available.

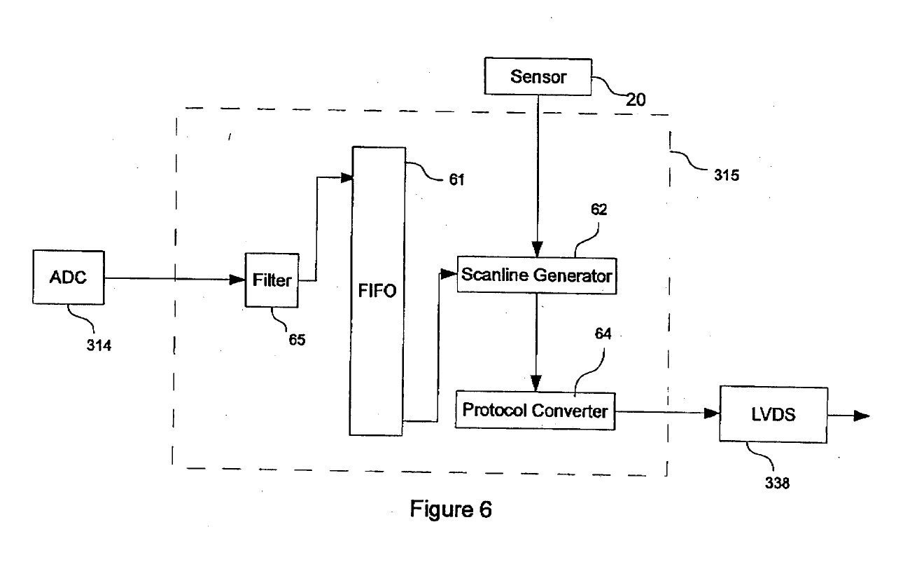

The scanline is generated in the controller 351. A partial block diagram of the functional blocks provided by the FPGA 315 is shown in FIG. 6. There is a FIFO buffer 61 which allows the scanlines to be asynchronously processed. Echo intensity data from the ADC data is received into the FIFO buffer via filter 65 and passed to a scanline generator 62. It is combined with orientation data from the orientation sensor 20 and has a CRC added for error correction over the data link. The data is then passed to a protocol converter 64 to be converted to a protocol suitable for transmission via the data channel. Any suitable protocol may be used. In this embodiment the protocol chosen for use on the data channel is 8b10b, which is well known in the art.

The 8b10b data is passed to an LVDS transmitter 338 and is transmitted via the data channel 320 to the DPU 11.

Referring to FIG. 3, the LVDS data channel is received by the DPU via LVDS receiver 321 and phase locked loop 322. The 8b10b data is passed to the DPU FPGA 341. Protocol conversion is performed by controller 352 to recover the original scanline data.

An application is now run by the DPU CPU 340 to process the scanlines for display as an ultrasound image on the display 16 of the DPU 11.

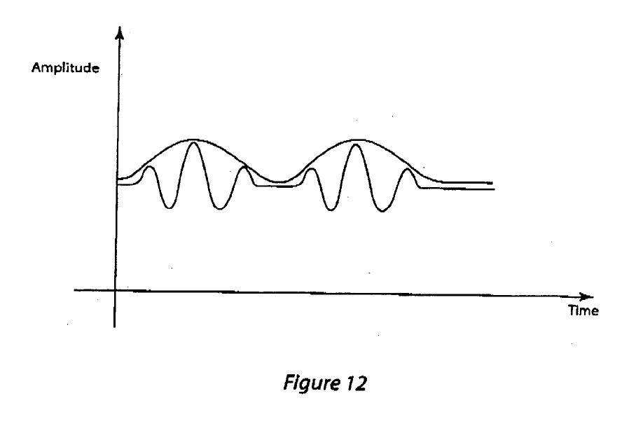

The scanline data at this point is still in the form as shown in FIG. 9b. This is not suitable for display. There is more information contained in the signal than can be displayed on a practical display. In order to provide scanlines with an information content compatible with display, an enveloping function is applied to each scanline, as shown in FIG. 12. Thus the raw scanline signal 123 is enveloped to produce a scanline which has the characteristics of the envelope 125. Any suitable enveloping function may be used. In an embodiment, a Hilbert transform is applied as the enveloping function.

The frequency of the enveloped data is less than that of the raw data signal allowing the enveloped data to be down sampled, that is, use fewer samples per time period than the raw signal, without loss of imaging information.

The application processes the scanlines in order to map the vector scanlines to a pixel buffer which may then be mapped to the physical pixels used by the display. Any suitable method of mapping vector data to a Cartesian grid may be employed. Interpolation is required in order to fill in pixels that do not coincide with scanlines.

Since no information concerning the linear displacement of the probe unit is sensed, all scanlines have a common, arbitrary origin. In embodiments where rotation about only one axis is sensed, the scanlines will also be co-planar in an arbitrary plane. In embodiments where rotation about more than one axis is sensed, it is necessary to choose a “plane of best fit” which will correspond to the plane of the display screen.

It is also necessary to choose a forward direction for the scan which will correspond to the vertical centreline of the screen display.

Any suitable method may be used to make these choices. In an embodiment, the forward direction is chosen by bisecting the angle which is the largest angle between any two scanlines.

The plane of best fit may be chosen by any means which minimises the degree to which scanlines deviate from the chosen plane. In an embodiment a mathematical process employing principal component analysis is undertaken to find this plane. The scanlines are then mapped to this plane.

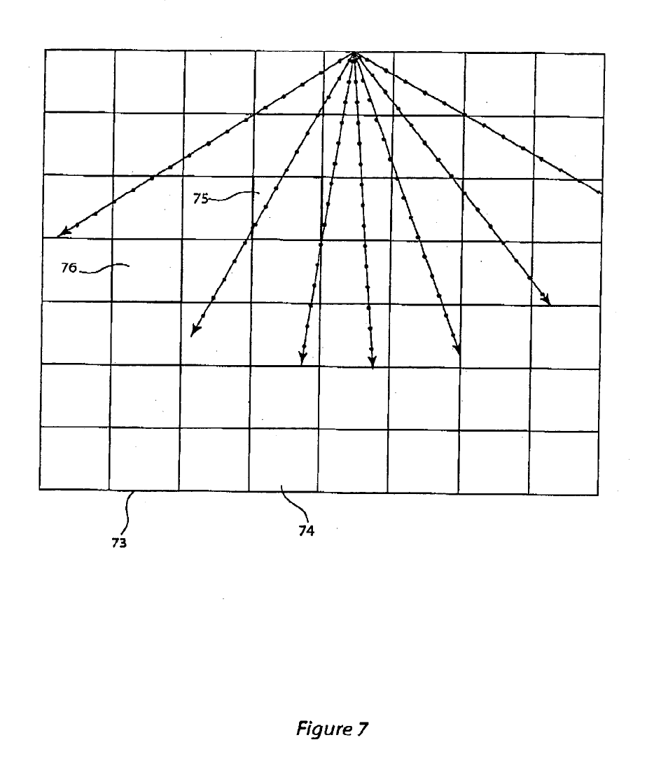

In a preferred embodiment a process we have called pixel row-wise scan interpolation is now applied to the scanline data to implement the process of mapping the scanlines to a pixel grid. As shown in FIG. 7, the scanline dataset is a series of scanlines 71, with a common origin. Each scanline consists of a number of data points 72. In the case of an ultrasound scan these are intensity of reflection values. For the purposes of display these are brightness values.

FIG. 7 also shows a pixel buffer pixel grid superimposed on the data. As can be seen, a display screen is a regular grid 73 of individual pixels 74. Each pixel can have only one brightness value. It can be seen that there are pixels 75 which are associated with more than one scan point and other pixels 78, which are associated with none. Pixel row-wise scan interpolation is applied to produce a data set with one and only one brightness value associated with each pixel.

Pixel row-wise interpolation begins by intersecting the scan lines with the pixel buffer one pixel row at a time.

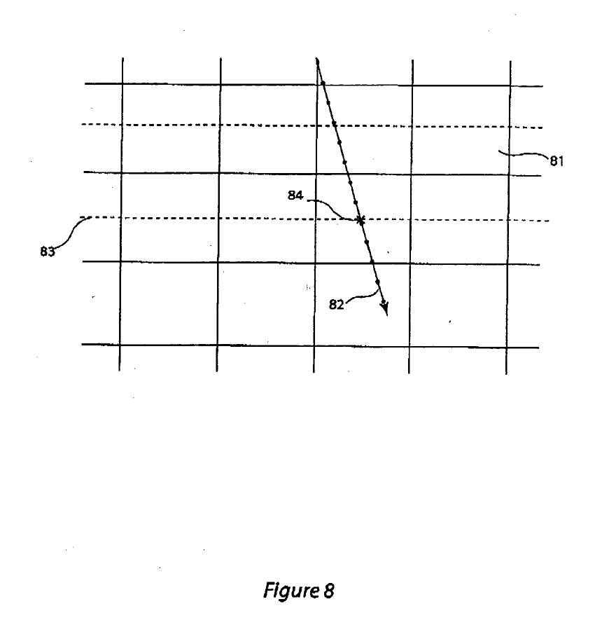

Looking at FIG. 8, there is a pixel row 81 and a scanline 82. We define a rowline 83 as the midline of the pixel row. There is one intersection point 84 between the rowline and the scanline.

Each of these intersection points is calculated for a given row. This gives an array of values sorted in the order of the received scanlines. This may not be the order of the column of the pixel grid. This can occur because the ultrasound probe unit, being hand scanned, may briefly wobble in a direction against the predominant direction of rotation, or indeed may have been swept back over already scanned areas by a user.

The calculated intersection points are now sorted into pixel column order, and order within each pixel.

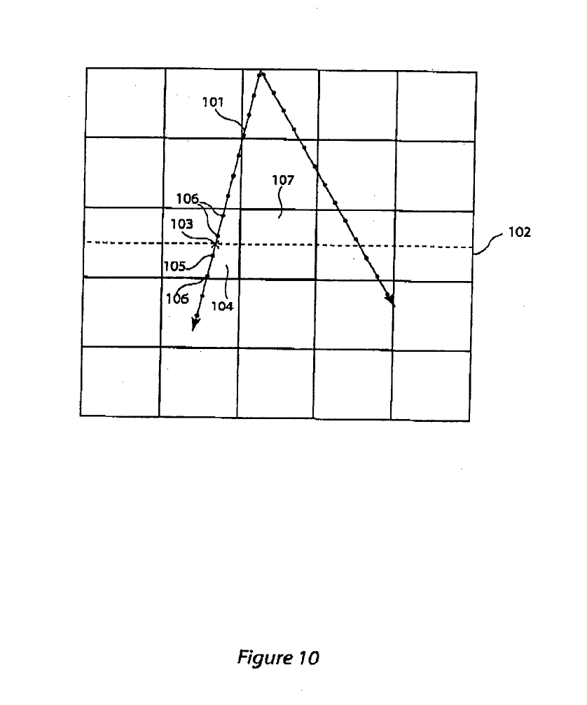

The value which is assigned to each pixel is chosen as that of the data point which is closest to the intersection point. This is shown on FIG. 10. Scanline 101 intersects rowline 102 at intersect point 103 in pixel 104. Scan data point 105 is closest to the intersection point and becomes the value for pixel 104. Scan data points 106, in the same pixel, are ignored and do not contribute to the displayed image.

There may be more than one intersect point in a pixel, when the angle between scanlines is sufficiently small that more than one scanline crosses a pixel. In this case, the pixel value is the mean of the value of the data points which are closest to each of the intersect points.

Also in FIG. 10, there are shown pixels 107 which are “holes”, that is they do not have a scanline intersect. In order to display a smooth image, these holes must be filled with values which are consistent with the filled pixels around them.

This is done by interpolation between pixels having defined values. Where linear interpolation is employed, the brightness value for the holes is defined such that there is a constant increment between the brightness values of the holes and adjacent pixels.

Other interpolation formulae may be used to fill in the values for the holes. The interpolation of the preferred embodiment is linear but quadratic, cubic or other higher order interpolations may be used.

Along each row, pixel values are interpolated between intersection points, which is computationally efficient as pixels along a row are contiguous in memory. Intersection points are computed and stored in fractional pixel index and fractional scan line index coordinates. After the first row of pixels, subsequent intersection points are determined simply by adding a constant offset to the fractional pixel and fractional scan line coordinates.

The result of this repeated processing is an array of values in the pixel grid buffer. These values are brightness values for the related pixel. This array is mapped to the physical pixels of display 16 and the result is a conventional ultrasound image where brightness corresponds to the intensity of echo, compensated for depth attenuation, and a picture of the internal features of the subject is formed.

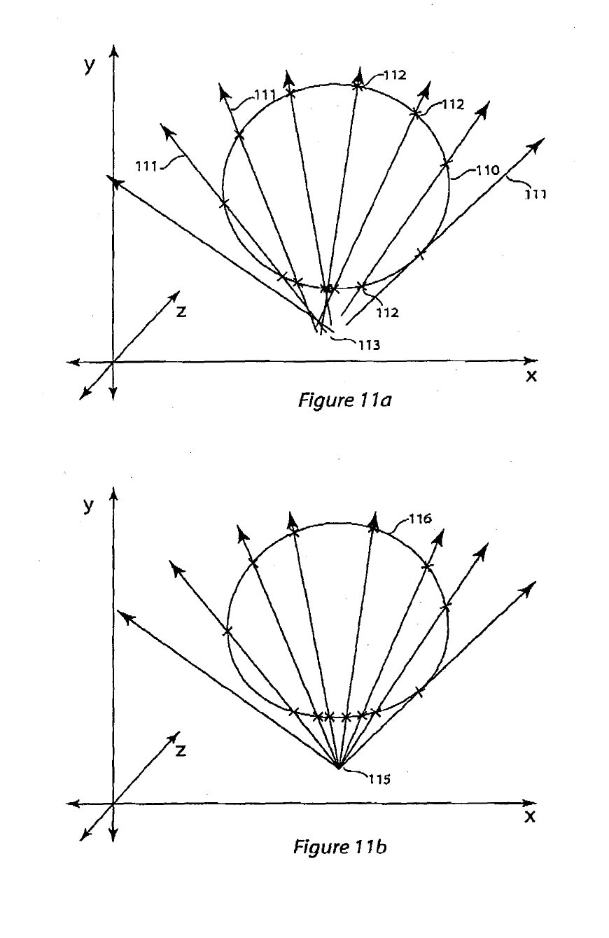

FIG. 11a shows a scanline dataset as it would be if all movement of the probe unit were able to be sensed. As illustrated in FIG. 11a, the origin for each of the scanlines will not actually be the same, despite the best efforts of the user to make it so. Some small displacement is likely to occur in each of the three spatial dimensions. There may also be some small rotation about axes other than the sensed axis or axes.

The prior art attempts to exactly map these origin points 113 in a fixed, external frame of reference.

However, we have discovered that very useful information can be obtained by neglecting these movements. By sensing only orientation, without sensing displacement, the origin points are inherently mapped to a single point.

The distortion of the resultant image caused by this is minimal, as illustrated in FIG. 11. FIG. 11a shows a perfectly circular target 110 which is isonified and scanned by a manual sweep as described above to produce scanlines 111.

In this idealised diagram, each scanline has zero intensity values, except at the points 112 where the target perimeter 110 is encountered.

Due to hand movement, the origin points 113 of the scanlines are not coincident.

However, if only the rotation of each scanline is measured, as shown in FIG. 11b, the scanlines are inherently mapped to a single origin point 115. If rotation about only a single axis is sensed, the scanlines are also inherently mapped to co-planarity. The angles and the intensity values of the scanlines are unaltered.

When the target perimeter scan points are joined, we have the scanned feature perimeter 116. As can be seen, the perimeter 116 is not perfectly circular, but the distortion is minimal.

In other embodiments, information sensed about errant rotation about an axis which is not the axis about which the user is attempting to rotate the probe unit can be made use of without using it to calculate a plane of best fit. In an embodiment, the magnitude of such rotation for each scanline is monitored by the CPU in the DPU. If the magnitude exceeds a selected value, which is calculated to introduce unacceptable distortion, the user is warned and the scan is not displayed. If the errant rotation is within acceptable limits, it is ignored, and the scanlines treated as if rotation about only a single axis has been sensed.

In an embodiment, the probe unit is shaped to assist the user to rotate the unit about only a single axis. This shaping may apply to the main body of the probe unit or to a transducer housing, or to both.

Although the invention has been herein shown and described in what is conceived to be the most practical and preferred embodiment, it is recognised that departures can be made within the scope of the invention, which is not to be limited to the details described herein but is to be accorded the full scope of the appended claims so as to embrace any and all equivalent devices and apparatus.

1. An ultrasound imaging system adapted for hand held use including:
a. a probe unit having a transducer in a fixed spatial relationship with the probe unit, the transducer being adapted to transmit and receive ultrasonic signals in substantially only one direction,
b. an orientation sensor configured to sense a rotation of the probe unit about at least one axis,
c. electronics configured to apply a pulsed electrical signal to the transducer and to process the electrical output signal of the transducer and of the sensor to produce scanlines, each scanline having a series of intensity values and a rotation value,
d. a processor configured to process the scanlines to produce a raster image,
e. a display configured to display the resultant raster image.

2. The system of claim 1 wherein the sensor is an inertial sensor.

3. The system of claim 1 wherein the sensor includes a gyroscope.

4. The system of claim 1 wherein the sensor includes two or more orthogonally mounted gyroscopes.

5. The system of claim 1 wherein the sensor includes an accelerometer.

6. The system of claim 1 wherein the sensor includes two or more orthogonally mounted accelerometers.

7. The system of claim 1 wherein the rotation is relative to a selected scanline.

8. The system of claim 7 wherein the selected scanline is the first scanline of a scan data set.

9. The system of claim 1 wherein the rotation is relative to the immediately preceding scanline.

10. The system of claim 1 wherein the processor is configured to map the scanlines to a plane of best fit when processing the scanlines to produce a raster image.

11. The system of claim 10 wherein the processor is further configured to map the scanlines to a pixel grid.

12. The system of claim 11 wherein the mapping includes pixel row-wise interpolation.

13. The system of claim 1 wherein the sensor is configured to sense rotation about at least two axes, with rotation about axes other than a selected axis being treated as errant rotation.

14. The system of claim 13 wherein a user is warned if the errant rotation exceeds a selected level, the selected level being chosen to limit distortion of the resultant image to a selected, acceptable level.

15. The system of claim 1 wherein the probe unit is shaped to assist a user to perform a sweep in which rotation about other than a selected axis is minimised.

16. A method of ultrasound imaging including the steps of
a. applying a probe unit to a target body, the probe unit including an ultrasound transducer configured to transmit and receive ultrasonic signals into and from the target body,
b. transmitting ultrasonic pulses into the target body and receiving return signals,
c. rotating the probe unit substantially in a single plane such that a two dimensional section of the target body is scanned,
d. providing a sensor at the probe, the sensor being configured to provide rotation information about the rotation of the probe unit about at least one axis,
e. receiving rotation information from the sensor,
f. combining the return signals with the rotation information to produce scanlines,
g. processing the scanlines to produce a raster image,
h. displaying the raster image on a display.

17. The method of claim 16 wherein the sensor is an inertial sensor.

18. The method of claim 16 wherein the sensor includes a gyroscope.

19. The method of claim 16 wherein the sensor includes two or more orthogonally mounted gyroscopes.

20. The method of claim 16 wherein the sensor includes an accelerometer.

21. The method of claim 16 wherein the sensor includes two or more orthogonally mounted accelerometers.

22. The method of claim 16 wherein the step of producing a raster image includes mapping the scanlines to a plane of best fit.

23. The method of claim 22 wherein the process further includes mapping the scanlines to a pixel grid.

24. The method of claim 16 wherein the step of producing a raster image includes applying row wise pixel interpolation to the scanlines.

25. The method of claim 16 wherein the probe unit is shaped to assist a user to rotate the probe unit substantially in a single plane.

26. The method of claim 25 wherein a transducer cover which is part of the probe unit is shaped to assist a user to rotate the probe unit substantially in a single plane.

27. A method of ultrasound imaging including:
a. providing a probe unit having a transducer configured to scan substantially only in a single direction at any instant,
b. providing an orientation detection sensor integral with the probe unit, and
c. combining an output of the transducer with the output of the sensor to produce diagnostically useful 2D tomographic images of a body to be scanned.

28-29. (canceled)
